(19) United States
(12) Patent Application Publication
(10) Pub. No.: US 2026/0316138 A1
(43) Pub. Date: Oct. 15, 2026

(54) METHODS AND APPARATUS FOR PATIENT-MATCHED SURGICAL IMPLANTS

(71) Applicant: Mighty Oak Medical, Inc., Englewood, CO (US)

(72) Inventors: George Frey, Cherry Hills Village, CO (US); Tyler Drumm, Littleton, CO (US); Caleb Voelkel, Fort Collins, CO (US); Jakob Yedo, Maple Grove, MN (US); Erick Solis, Wheat Ridge, CO (US)

(73) Assignee: Mighty Oak Medical, Inc., Englewood, CO (US)

(21) Appl. No.: 19/631,858

(22) Filed: Mar. 27, 2026

Related U.S. Application Data
(60) Provisional application No. 63/779,173, filed on Mar. 27, 2025.

(51) Int. Cl. B21D 7/04; A61B 17/56; A61B 17/70; B21F 45/00

(52) U.S. Cl. CPC B21D 7/04; B21F 45/008; A61B 2017/568; A61B 17/7002

(57) ABSTRACT

Apparatus for bending a rod are disclosed. The apparatus may include one or more of several components, including a bending die, wherein the bending die is configured to rotate about at least one axis; at least one bending die head; at least one bending die head housing, wherein the die head housing includes a translational element about its x-axis; and, an insert that fits around and is secured to the bending die. Methods of operating the apparatus are also disclosed.

### CROSS REFERENCE TO RELATED APPLICATIONS

This application claims the benefit of and priority under 35 U.S.C. § 119(e) to U.S. Provisional Patent Application Ser. No. 63/779,173 filed on Mar. 27, 2025, the entirety of which is incorporated herein.

The following applications are commonly owned by Applicant and are incorporated herein by reference: U.S. patent application Ser. No. 18/502,667, filed on Nov. 6, 2023; U.S. patent application Ser. No. 17/402,512, filed on Aug. 14, 2021, now issued as U.S. Pat. No. 11,806,197; U.S. patent application Ser. No. 16/831,215, filed on Mar. 26, 2020, now issued as U.S. Pat. No. 11,633,254; U.S. patent application Ser. No. 16/598,861, filed on Oct. 10, 2019; U.S. patent application Ser. No. 15/997,404, filed Jun. 4, 2018; U.S. patent application Ser. No. 15/416,975, filed on Jan. 26, 2017, which issued as U.S. Pat. No. 9,987,024 on Jun. 5, 2018; U.S. patent application Ser. No. 14/883,299, filed Oct. 14, 2015, which issued as U.S. Pat. No. 9,642,633 on May 9, 2017; U.S. patent application Ser. No. 14/298,634, filed Jun. 6, 2014, which issued as U.S. Pat. No. 9,198,678 on Dec. 1, 2015; U.S. patent application Ser. No. 13/841,069, filed Mar. 15, 2013, which issued as U.S. Pat. No. 8,870,889 on Oct. 28, 2014; and U.S. patent application Ser. No. 13/172,683, filed Jun. 29, 2011, which issued as U.S. Pat. No. 8,758,357 on Jun. 24, 2014.

### FIELD OF THE INVENTION

The present disclosure generally relates to the field of medical devices, and more specifically towards methods and apparatus for using and fabricating patient-specific or patient-matched surgical devices, such as implants, based in part on the patient's unique anatomical features.

### BACKGROUND OF THE INVENTION

Given the complexities of surgical procedures and the various tools, instruments, implants and other devices used in the procedures, as well as the varying anatomical differentiation between patients who receive those tools, instruments, implants and devices, it is often challenging to create a surgery plan that accounts for the unique and sometimes irregular anatomical features of a particular patient.

For example, the implantation of rods, screws or other fixation devices in a patient's boney anatomy is well accepted amongst surgeons who treat various orthopedic pathologies. Although the performance of various rods and screw constructs has become predictable, there are still multiple challenges with the placement and insertion of these fixation devices. The challenges occur, for example, when a surgeon is unable to reference boney landmarks due to previous surgery or when the patient's anatomy is irregular in shape, or when a particular placement of rods, or the insertion of screws (or other fixation devices) is impeded by anatomical obstructions. Similar challenges exist when configuring rods, screws or other implants in a desired orientation for correction of a patient's spine.

Surgeons now have the ability to readily convert magnetic resonance imaging (MRI) data or computed tomography (CT) data into a data set readable by computer-aided design (CAD) program and/or finite element modeling (FEM) program, which then may be used to create, for example, a customized surgical guide and/or implant based on the dynamic nature of the anatomical structures the customized guide/implant is designed to associate with. This data, while currently used by surgeons in surgery planning, is largely unused for creating a customized set of implants or other surgical devices that are designed to complement the patient's unique anatomy.

Specific surgical procedures are often performed in the spinal region of a patient. The procedures performed in these areas are often designed to stop and/or eliminate all motion, including by removal and/or destruction of some or all of the boney anatomy in the patient's boney anatomy and/or implantable fixation devices for limiting movement of the boney anatomy of the particular patient. By eliminating movement, pain and degenerative disease may be reduced or avoided.

A significant danger of performing operations on a patient's orthopedic anatomy, and in particular accessing an intervertebral space during a MIS surgery on the spine, is that of inadvertently contacting or damaging the para-spinal nerves, including the exiting nerve roots, traversing nerves and the nerves of the cauda equina. The exact location of these para-spinal nerves cannot be precisely determined prior to the commencement of surgery, and therefore are dependent on a surgeon's ability to visually locate the same after the initial incision is made. Moreover, intervertebral spaces in the spine have other sensitive nerves disposed at locations which are not entirely predictable prior to insertion of the surgical tool into the intervertebral area. Accordingly, the danger of pinching or damaging spinal nerves when accessing an intervertebral space has proven to be quite limiting to the methods and devices used during minimally invasive spinal surgery.

In addition, as cannula are received through the patient's back, such as when performing minimally invasive spinal surgery, minor blood vessels are ruptured, thereby blocking the surgeon's vision inside the intervertebral region after the cannula has been inserted. Other anatomical features at a particular patient may also obstruct the surgeon's view or make it difficult to provide illumination within the cannula. Therefore, one particular shortcoming that is addressed by the present disclosure is to provide devices which are patient-matched to facilitate proper location and orientation without use of microscopes or other equipment and that otherwise eliminate the problems associated with prior art procedures on the spine, including MIS procedures.

The prior art also fails to teach a system for creating patient-specific or patient-matched surgical apparatus, based on the data set derived from the MRI or CT scan, for use with robotic and augmented reality (“AR”) systems. The use of the patient-specific data set for a vertebral or other anatomic body of a particular patient may allow a surgeon to accommodate for subtle variations in the position and orientation of an instrument, screw, plate or other bone anchor to avoid particular boney anatomy or irregularities in the positioning and alignment of the adjoining vertebral bodies.

As another example, the use of these data sets may also assist a surgeon in selecting and achieving a desired curvature, for example in a rod, to correct an anomaly or deformity. Similar problems exist with respect to placement of rods, screws and other implants so as to avoid sensitive anatomical features of a particular patient, or to secure a bone anchoring device in a particular area of desired bone density during an actual procedure. The use of patient-specific data sets also permit the surgeon to create a patient-contacting surface that is oriented to match one or more of the anatomical features represented by the data set, and thereby quickly and efficiently locate and place the patient-contacting surface(s) in the appropriate location and orientation.

It would therefore be advantageous to provide a patient-specific apparatus that is adapted to conform to a plurality of anatomical features of a particular patient and that otherwise assists a surgeon in completing the surgical procedure(s) safely and efficiently. It is also advantageous to provide a procedure and/or apparatus that otherwise significantly reduces, if not eliminates, the problems and risks noted above. Other advantages over the prior art will become known upon review of the Summary and Detailed Description of the Invention and the appended claims.

### SUMMARY OF THE INVENTION

According to one aspect of the present disclosure, a novel system and method is described for developing customized apparatus for use in one or more surgical procedures, particularly those procedures associated with the cervical, lumbar and/or thoracic areas of the spine.

There are various types of spinal disorders that may cause an abnormal state for the patient, including by way of example but not limitation: hyper/hypo lordosis (lumbar or cervical spine); hyper/hypo kyphosis (thoracic or cervical spine); scoliosis (coronal plane curvature); spondylolisthesis; and other disorders. Patients that suffer such conditions frequently experience extreme pain, radiating pain, and reduced nerve function. These patients are typically treated via a posterior spinal fusion procedure, often involving the introduction of one or more rods to stabilize these segments, decompress nerves, and bring the patient's spine back into global balance.

During such procedures there are several potential abnormal states to address. For example, correction of spinal deformities often includes at least one, if not more, of the following: curvature correction, derotation of vertebrae, sagittal and/or coronal balance restoration, prevention of further deformity, improvement or preservation of neurological function, and curvature stabilization or reduction. As these objectives are complex and difficult to achieve, a need exists for three-dimensional patient-specific rod that provides a contour that corresponds to a spinal curvature that is healthy and achievable in the patient to help achieve the planned surgical objectives.

The systems and methods described herein incorporate a patient's unique morphology, which may be derived from capturing MRI, CT, or other data to derive one or more “Patient Matched” apparatus, which comprises complementary surfaces based on a plurality of data points from the MRI, CT or other anatomical data. Each “Patient Matched” apparatus is matched and oriented around the patient's own anatomy, and is preferably configured to incorporate specific and/or desired insertional trajectories (which may be verified in a pre-operative setting using 3D CAD software, such as the software disclosed in WO 2008027549, which is incorporated by reference herein in its entirety). According to one embodiment described herein, other apparatus used during the surgical procedure may facilitate the orientation and/or placement of one or more instruments, implants, including rods, plates, screws, fixation devices, etc.

The patient-specific apparatus described herein may interface with any one or multiple vertebra levels, including without limitation the cervical, lumbar, pelvis, and thoracic areas of the spine. In one aspect, the apparatus are formed as described in Appendix A, which is expressly incorporated herein by reference in its entirety.

Current rod contouring methods and contoured rods are bent intraoperatively by the surgeon attempting to achieve ideal spinopelvic parameters for the patent by relying upon the surgeon's dexterity and expertise. However, research shows that intraoperative contouring of the rod by the surgeon is not always accurate and may rely upon trial and error to obtain the necessary curvature, which extends operating time and may weaken the rod and a rod with a non-ideal curvature may be implanted.

The present disclosure aims to resolve issues with intraoperative rod contouring by the surgeon by creating a pre-operative 3D virtual model, and modifying the model to an achievable correction that achieves surgical objectives. From the operative plan virtual model, the location(s) that the rod needs to be in, to hold the necessary curvatures and be fixated to other fixation elements, may be effectively planned and implemented.

In order to create a manufacturable rod that is able to hold the 3D curve and be fixed to fixation elements, the contour of the spine that would be created needs to be simplified. This invention describes a method of manufacturing said simplified, manufacturable rod and the apparatus used to execute this method of manufacturing.

By way of providing additional background, context, and to further satisfy the written description requirements of 35 U.S.C. § 112, the following are incorporated by reference in their entireties for the express purpose of explaining and further describing the various tools and other apparatus commonly associated therewith surgical procedures: U.S. Pat. No. 6,309,395 to Smith et al.; U.S. Pat. No. 6,142,998 to Smith et al.; U.S. Pat. No. 7,014,640 to Kemppanien et al.; U.S. Pat. No. 7,406,775 to Funk, et al.; U.S. Pat. No. 7,387,643 to Michelson; U.S. Pat. No. 7,341,590 to Ferree; U.S. Pat. No. 7,288,093 to Michelson; U.S. Pat. No. 7,207,992 to Ritland; U.S. Pat. No. 7,077,864 Byrd III, et al.; U.S. Pat. No. 7,025,769 to Ferree; U.S. Pat. No. 6,719,795 to Cornwall, et al.; U.S. Pat. No. 6,364,880 to Michelson; U.S. Pat. No. 6,328,738 to Suddaby; U.S. Pat. No. 6,290,724 to Marino; U.S. Pat. No. 6,113,602 to Sand; U.S. Pat. No. 6,030,401 to Marino; U.S. Pat. No. 5,865,846 to Bryan, et al.; U.S. Pat. No. 5,569,246 to Ojima, et al.; U.S. Pat. No. 5,527,312 to Ray; and U.S. Pat. Appl. No. 2008/0255564 to Michelson.

According to one aspect, embodiments of the invention comprise: a machine or apparatus for bending a rod, comprising: at least one die, wherein the at least one die is configured to rotate about at least its y-axis; at least one die head; at least one die head housing for each of the at least one die heads; wherein the die head housing includes a translational element about its x-axis; and, an insert that fits around and is secured to the at least one die.

In another aspect, the machine or apparatus described herein may comprise at least one second die, at least one second die head, and/or at least one second die head housing.

According to another aspect, embodiments of the invention comprise: a machine for bending a rod, comprising: one bending die, wherein the bending die can rotate about at least its y-axis; at least one bending head; a bending head housing for each of the at least one bending heads; wherein the bending head housing includes a rotational element about its x-axis; wherein the bending head housing includes a translational element along the y-axis; wherein the bending head housing includes a translational element along the x-axis; an insert that fits around and is secured to the bending die; wherein the insert comprises a track for receiving and/or retaining a surgical implant, such as a rod.

According to yet another aspect, embodiments of the invention comprise: a machine or apparatus for bending a rod, comprising: at least one bending die, wherein the at least one bending die can rotate about at least its y-axis; at least one bending head; wherein the at least one bending head has a substantial y-axis height compared to the x-axis dimension; a bending head housing for each of the at least one bending heads; wherein the bending head housing includes a translational element about its x-axis; an insert that fits around and is secured to the bending die; and, wherein the substantial y-axis height of the bending head allows for the insert to have a track for the rod that is in multiple planes.

According to yet another aspect, a robotic apparatus may control the position and orientation of each motion element and may be programmed to induce the designed bend or curvature profile of the implant, including the curvature of a rod. In this scenario, each element may be controlled simultaneously and synchronized with the other motion elements. Alternatively, each motion element may be controlled manually and moved to a pre-set position and orientation in order to induce the designed bend. Alternatively, the bending head rotational element about its x-axis and the bending head translational element along the y-axis may allow motion through freedom of motion and lack of mechanical constraints in the design. The motion of these elements may be driven based on a designed interaction with the insert (i.e. grooves) or alignment with the rod. Alternatively, some combination of robotic, manual, and freedom of motion may be used to control the position and orientation of each motion element in order to induce the designed bend.

According to other aspects, the invention comprises an insert that is additively manufactured or CNC manufactured. The insert may be developed to correspond to a presurgically planned rod wherein the insert shape and sequence of bends produce the presurgically planned rod to correspond to a presurgical correction plan of the patient's spine. Said presurgically planned rod may be deigned to have multi-planar or uniplanar bends wherein each bend comprises of a curve defined by one of, but not limited to, a standard radius, a polynomial, a Bezier curve, an Archimedes' spiral, a line, or an ellipse. The insert and track are planned such that following the bending process and spring-back, the rod produced by the bending process matches said presurgically planned rod and is not notched, scratched, or otherwise blemished.

According to yet another aspect, embodiments of the invention comprises: a machine for bending a rod, comprising: a bending die, wherein the bending die can rotate about at least its y-axis; at least two bending heads; a bending head housing for each of the at least two bending heads; wherein each of the at least two bending head housings include a translational element about its x-axis; an insert that fits around and is secured to the bending die; wherein the position of the bending heads are controlled in relation to one another on the XZ plane.

The positional control of the bending heads in relation to one another allows for the heads to be kept at a specified distance from one another regardless of the radius of the insert or the rod being bent. This allows for a more reliable bend following spring-back.

According to yet another aspect, the bending die may rotate about its y-axis or another axis that causes the bending heads to move along the surface of the bending die and insert.

According to yet another aspect of the present disclosure, the bending die may be rotationally fixed and the one or more bending arm(s) and bending arm housing(s) rotate about the Y-axis or other axis of the bending die.

Another aspect of the present disclosure is to provide a method for preparing a customized surgical device or instrument, which in a preferred embodiment comprises, but is not limited to: (1) obtaining data associated with a patient's anatomy; (2) converting the data obtained to a 3-dimensional data set(s); (3) determining at least one trajectory or path for facilitating a surgical procedure to be performed on the patient; (4) determining at least one surface associated with the patient's anatomy; (5) generating a 3-dimensional representation of the customized surgical device or instrument, which incorporates the at least one trajectory of path and a matching surface to the at least one surface associated with the patient's anatomy; (6) fabricating the customized surgical device or instrument using the 3-dimensional representation; (7) registering at least one marker on the customized surgical device with a robotic or an AR system; and (8) positioning the customized surgical device on the patient's anatomy utilizing the at least one surface associated with the patient's anatomy and the at least one marker. Other aspects of the method(s) are described in Appendix A hereto.

According to this aspect described above, the method steps may further comprise adjusting the size of the modeled device to accommodate the space limitations on the surgeon, orienting elements of the modeled device to avoid certain anatomical features, creating one or more surfaces that may conveniently be operatively associated with one or more instruments and/or tools used in the surgical procedure(s), etc.

According to yet another aspect of the present disclosure, the system and method includes use of data obtained from a radiographic imaging machine, an ultrasonic machine, a bone density scanning machine, or a nuclear medicine scanning device.

In another aspect, the patient-matching features may be confirmed by one or more additional process, such as fluoroscopy or other processes known to those of skill in the art.

In one aspect of the present disclosure, the method comprises the use of bone density data obtained through a CT scan of the patient anatomy for use in planning the trajectory of a surgical guide and corresponding fixation device or instrument, such as a cutting/routing/drilling instrument intended to penetrate the boney anatomy. This data may be used in other manners contemplated and described herein to assist the surgeon in planning, visualizing or otherwise preparing for the surgical procedure for the patient.

In yet another alternative embodiment, the data obtained from one of the scanning devices described above may be supplemented or merged with data from a bone density scanner to fabricate a device that is designed to remain in the patient after the surgical procedure is completed. It is to be expressly understood that data from a bone density scanner is not necessary to practice the inventions described herein, but may supplement the data and assist a surgeon or other medical professional in determining the proper location, trajectory, orientation or alignment of the various apparatus described herein.

According to yet another aspect of the present disclosure, data may be supplemented or merged with data from a bone density scanner to achieve further control over the orientation of any desired axes, particularly where the surgical procedure involves insertion of one or more implantable devices.

According to yet another embodiment, the data obtained from the patient permits the apparatus to be manufactured with defined pathways through the apparatus, which are operatively associated with at least one tool, instrument, or implant, and which permit the at least one tool, instrument or implant to be inserted in the defined pathways in a consistent and reproducible manner. Examples of devices that are implanted or remain in the patient include anchoring devices such as screws, pins, clips, hooks, etc., and implantable devices such as spacers, replacement joints, replacement systems, cages, etc. The apparatus may comprise one or more stops located within the pathways for preventing a tool, instrument or implant from advancing beyond a predetermined distance.

In one embodiment, the rod may have different diameters in different sections of the rod. In one embodiment, at least a first portion of the rod may be directed in a first predetermined trajectory. In another embodiment, the rod may comprise both a first and a second predetermined trajectory. In another embodiment, portions of the rod may be directed in a plurality of trajectories, each different from the others.

The Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention and no limitation as to the scope of the present disclosure is intended by either the inclusion or non-inclusion of elements, components, etc. in this Summary of the Invention. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the claims set forth herein below define the invention.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. For further illustration, the information and materials supplied with the provisional and non-provisional patent applications from which this application claims priority are expressly made a part of this disclosure and incorporated by reference herein in their entirety.

Additionally, although the rods described above are particularly well-suited for implantation into the spinal column, and although much of the discussion of the present disclosure is directed toward their use in spinal applications, advantages offered by embodiments of the present disclosure may also be realized by implantation at other locations within a patient where correction or realignment of bony structures is desired. As one of skill in the art will appreciate, the present disclosure has applications in the general field of skeletal repair and treatment, with particular application to the treatment of spinal injuries and diseases. It should be appreciated, however that the principles of the present disclosure can also find application in other areas.

It is expressly understood that where the term “patient” has been used to describe the various embodiments of the disclosure, the term should not be construed as limiting in any way. For instance, a patient could be either a human patient or an animal patient, and the apparatus and methods described herein apply equally to veterinary science as they would to surgical procedures performed on human anatomy. The apparatus and methods described herein therefore have application beyond surgical procedures used by spinal surgeons, and the concepts may be applied to other types of “patients” and procedures without departing from the spirit of the present disclosure.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

The present inventions, in various embodiments, include components, methods, processes, systems and/or apparatuses substantially as depicted and described herein, including various embodiments, subcombinations, and subsets thereof. Those of skill in the art will understand how to make and use the present inventions after understanding the present disclosure. The present inventions, in various embodiments, include providing devices and processes in the absence of items not depicted and/or described herein or in various embodiments hereof, including in the absence of such items as may have been used in previous devices or processes, e.g., for improving performance, achieving ease and\or reducing cost of implementation.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures. In the drawings:

FIG. 1 is a perspective view of an apparatus according to one embodiment of the present disclosure;

FIG. 2 is a partial plan view of an apparatus according to one embodiment of the present disclosure;

FIG. 3 is a detailed view of portions of the apparatus according to embodiments of the present disclosure;

FIG. 4 includes a perspective view of an insert for the apparatus according to embodiments of the present disclosure;

FIG. 5 includes a plan view of an insert according to another embodiments of the present disclosure;

FIG. 6 includes another plan view providing additional details of embodiments of the present disclosure;

FIG. 7 is a detailed plan view of a bending die and insert according to embodiments of the present disclosure;

FIG. 8 is a plan view of an alternate embodiment of the present disclosure; and

FIG. 9 is another plan view of the embodiment of FIG. 8.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

The human spine is a complex system of bones and connective tissue. The spine includes vertebral bodies that sit atop one another to support the body and protect delicate neural elements including nerves and the spinal cord. Between each vertebral body is a intervertebral disc that provides cushion and dampening for compressive forces. The spine has a natural curvature that when brought to an abnormal curvature or other diseased state via degeneration, trauma, or deformity can cause global imbalance, pain, and the inability of the spine to perform as intended.

There are various types of spinal disorders that may cause an abnormal state for the patient, including by way of example but not limitation: hyper/hypo lordosis (lumbar or cervical spine); hyper/hypo kyphosis (thoracic or cervical spine); scoliosis (coronal plane curvature); spondylolisthesis; and other disorders. Patients that suffer such conditions frequently experience extreme pain, radiating pain, and reduced nerve function. These patients are typically treated via a posterior spinal fusion procedure, often involving the introduction of one or more rods to stabilize these segments, decompress nerves, and bring the patient's spine back into global balance. During such procedures there are several potential abnormal states to address. For example, correction of spinal deformities often includes at least one, if not more, of the following: curvature correction, derotation of vertebrae, sagittal and/or coronal balance restoration, prevention of further deformity, improvement or preservation of neurological function, and curvature stabilization or reduction.

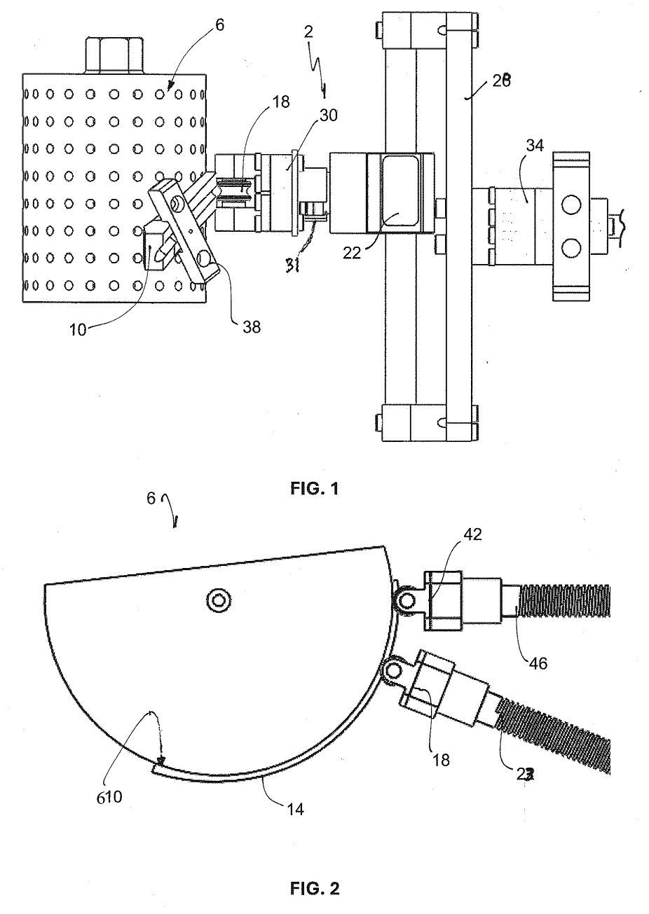

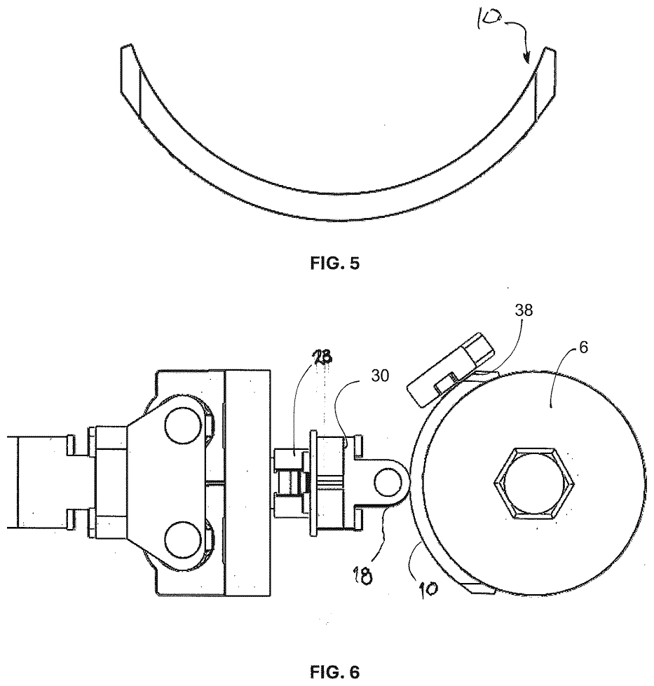

Embodiments of the present disclosure are shown in FIGS. 1-9 appended hereto. According to preferred embodiments, the present disclosure relates to a machine or apparatus 2 for bending a rod or other implant, and preferably comprises: a bending die 6; wherein the bending die 6 can rotate about its y-axis; at least one bending head 18; and, a housing 30 for each of the at least one bending head(s) 18. In a preferred embodiment, the housing includes a rotational element about its x-axis. In another preferred embodiment, the housing includes a translational element along the y-axis.

Referring to FIG. 1, a bending die 6 is shown that is substantially cylindrical. The bending die 6 may rotate about an axis, such as an axle positioned within the bending die 6. At least one bending head 18 may be coupled to a housing 30, as shown. The housing 30 may further be coupled to a rotational element 31 to permit rotation about an x-axis. The housing 30 and bending head 18 may further be coupled to a translational assembly 28 comprising a rotational element 22. The translational assembly 28 may be coupled to a second housing 34.

Referring to FIG. 2, a partial view of the apparatus 2 including the bending die 6 is shown in plan view. Here, a first bending head 18 and a second bending head 42 are shown coupled to first bending head assembly 23 and second bending head assembly 46. Each of the bending heads 18, 42 imparts a bend to a rod 14 by squeezing the rod 14 between the bending heads 18, 42 and the outer circumference 610 of bending die 6.

Referring back to FIG. 1, and according to embodiments, the apparatus 2 may also comprise an insert 10 that fits around and pressed against the bending die 6. In a preferred embodiment, the insert 10 has a track 181 for a rod to be inserted or received, and is configured to introduce a bend to the rod as best shown in FIG. 4. More specifically, the rod may be received by the track 181 and biased against the bending head(s) 18, 42, which causes bending of the rod as the bending die 6 rotates. In embodiments, a clamp 38 may be employed to secure the rod in place during operation and bending of the rod. In another embodiment, a second bending head 42 may be employed to secure the rod in place during operation and bending of the rod.

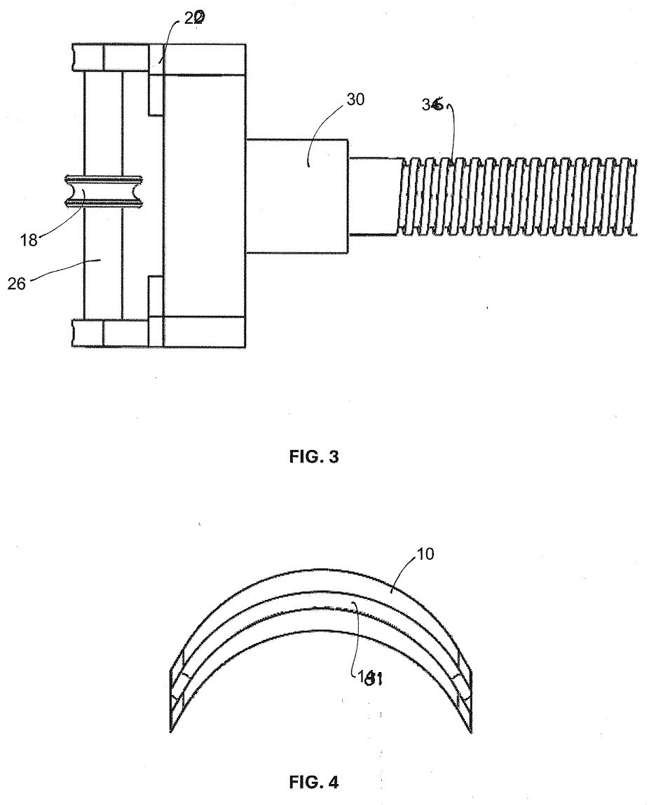

According to embodiments, the insert 10 may be additively manufactured, CNC manufactured, and/or may be customized to a specific patient-profile (i.e., specific to the proposed correction for a specific patient). A detailed view of the insert 10 is shown in FIG. 4. A detailed view of the bending head 18 and housing 30 is shown in FIG. 3. The bending head 18 may rotate about an axle 26, and be coupled to housing 30 by one or more fixation members 20. The bending head assembly 23 may comprise a threaded portion 36 to introduce further translational movement of the bending head assembly.

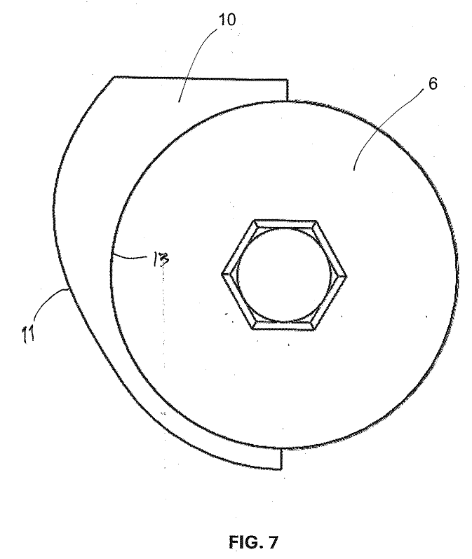

Referring now to FIG. 4, a detailed view of an insert 10 is shown according to a one embodiment. The insert 10 generally has an inner or first surface that is configured to mate with the outer circumference of the bending die 6. The outer or second surface, however, may vary depending on the type of bending radius or complexity of the bend imparted on the implant, as described below. Turning to FIG. 7, a bending die 6 is shown with a complex insert 10, which comprises a first surface 13 and a second surface 11. The second surface 11 may be contoured or configured to impart a specific bend to a rod, for example, depending on the nature of the patient's deformity or other abnormality. By further way of example but not limitation, the insert 10 may be customized for a specific patient, and incorporate patent-matched data to produce a rod with the appropriate bend(s) and contours required for the specific patient. In other embodiments, the second surface 11 may be configured for a particular length of rod, or to impart a larger bend in a specific portion of the rod.

Variations on the nature and complexity of the insert 10 are contemplated beyond those examples provided here. The customized and integrated matching aspects of this presently disclosed system provides an advantage over the prior art, in particular by providing a plurality of matching surfaces and/or matching points for each apparatus, which are easily or efficiently registerable and positionable. These advantages in turn reduce the likelihood of misalignment, misplacement and subsequent mistake during the surgical procedure(s). For example, according to embodiments, the x-position, y-position, and/or rotational position of the bending head is controlled by an actuator.

FIG. 6 shows additional details of the embodiment of FIG. 1. Here, the apparatus 2 may further comprise a clamp 38, which is positioned over the second surface of the insert 10 to further compress and retain the rod against the bending die 6 during operation of the apparatus 2. The clamp 38 is preferably adjustable and may be removed with ease once a bending operation is completed. In FIG. 6, the housing 30 and rotational element 28 are also depicted.

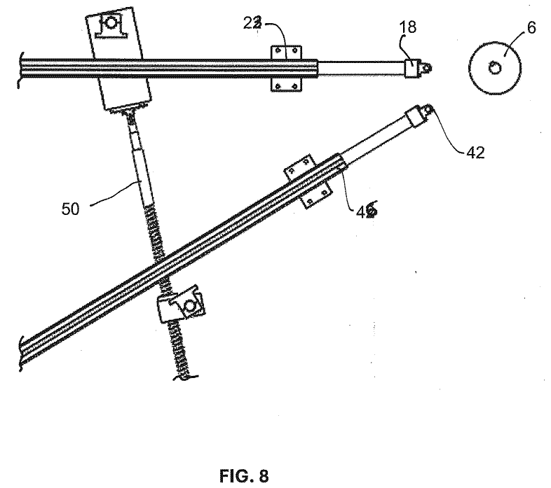

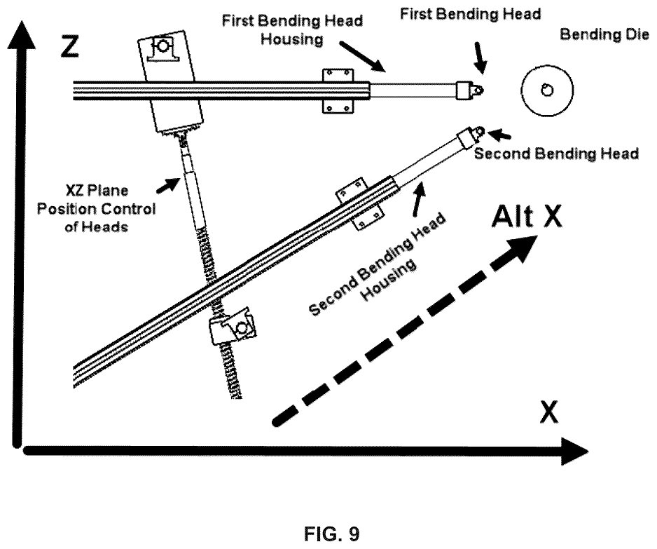

In FIGS. 8-9, another embodiment is shown. Here, a first bending head assembly 23 and a second bending head assembly 46 are used in conjunction with a bending die 6. The two assemblies 23, 46 may be selectively joined together by a position control device 50, which may be adjusted in the xz-plane. For example, the position control device 50 may be coupled to the first bending head assembly 23 and coupled to the second bending head assembly 46. Next, the position control device 50 may have an adjustment feature 50 may have a threaded portion that is selectively positioned in a threaded sleeve. By threading (or unthreading) the threaded portion relative to the threaded sleeve, the positions of the first and second bending head assemblies 23, 46 may be adjusted. A first bending head 18 and a second bending head 42 may impart different forces on the implant, such as a rod, while it is biased against the outer circumference of the bending die 6.

In alternate embodiments, the bending die 6 may be configured to translate along the y-axis. Therefore, the translational element along the y-axis of the housing 30 may be eliminated. In yet another embodiments, the bending die 6 may be configured to translate along the x-axis. Therefore, the translational element along the x-axis of the housing 30 may be eliminated. In yet another alternate embodiment, the bending die 6 may be configured to rotate about its x-axis, such that the housing 30 does not need to include a rotational element about its x-axis. And finally, the housing 30 may be configured to include a rotational element about the y-axis such that the bending die 6 does not need to rotate about its y-axis.

The invention may further comprise fixturing or a second bending head 42 to hold the rod in place during bending. The rod may be bent by the bending head 42 being forced against the rod and the rod being forced into the insert 10 and the bending die 6 rotating. Multiple inserts 10 may be utilized to facilitate multiple bends. Insert is used to generate bend, then the rod containing bend may be removed and re-fixed to the bending die 6 before being bent again with insert to generate bend. This can be done to apply multiple, successive bends in the same plane of the rod, or the rod may be rotated between bends in order to generate multi-planar bends in the rod or bends in the opposite direction in the same plane.

In embodiments, the rotation of the bending die 6 is controlled by an actuator. The apparatus 2 may also be configured to bend uniplanar rods. In embodiments, the bending head 18 may be prevented from moving backwards in the x-direction by a spring or hydraulic mechanism, or a hard stop. In another embodiment, the housing does not include a translational element along the y-axis, or does not include a rotational element about its x-axis.

In a preferred embodiment, housing includes a rotational element about its x-axis. In another preferred embodiment, housing includes a translational element along the y-axis. In certain embodiments, housing includes a translational element along the x-axis, as in FIGS. 8-9.

In yet additional embodiments, a second bending die and second housing may be configured for use with a first bending die and first housing to impart multiple bends along a rod.

The rod may be cylindrical. Alternatively, the rod is not cylindrical.

Methods of fabricating and using the apparatus described herein are also contemplated. Various steps may be included in the method of use, including but not limited to: manufacturing an insert with a track; securing the insert to a bending die; placing a rod at a proximal end of the track of the insert; clamping the rod in place; advancing a bending head within a housing in the x-direction until the bending head contacts the rod; preventing the bending head within the housing from moving backwards in the x-direction; and turning the bending die about its y-axis.

In other embodiments, the method may be used to form a multi-planar fixed radius rod. In this embodiment, the steps may further include: manufacturing an additional insert with a track, which takes into account the desired bend in rod about a rotated plate; securing an additional insert with a track to a bending die; rotating the rod so existing bend matches bend in additional insert's track; placing the rod in the additional insert's track; clamping the rod in place by a clamp; advancing the bending head within the housing in the x-direction until contacting the rod; preventing the bending head from moving backwards in the x-direction; turning the bending die about its y-axis; translating the bending head within the housing along the y-axis and rotating about its x-axis to maintain position relative to track in insert; and removing multi-planar bent rod from additional insert's track.

A method for fabricating a uniplanar variable radius rod is also disclosed. In this embodiment, the method may further comprise the steps of: manufacturing an insert with a track that includes a variables radius; securing the insert to a bending die; placing the rod at a proximal end of the track; clamping the rod in place by a clamp; advancing the bending head within the housing in the x-direction until contacting the rod; retreating the bending head in the x-direction to maintain contact of rod to insert; turning the bending die about its y-axis.

In other embodiments, a method for fabricating a multi-planar variable radius rod is disclosed. In this embodiment, the steps may further include: manufacturing an additional insert with a track, which may comprise a variables radius, taking into account the bend in rod and in the rotated plate; securing an additional insert with a track to a bending die; rotating the rod so existing bend matches bend in additional insert's track; placing the rod in the additional insert's track; clamping the rod in place by a clamp; advancing the bending head within the housing in the x-direction until contacting the rod; preventing the bending head from moving backwards in the x-direction; turning the bending die about its y-axis; advancing the bending head and retreating in the x-direction to maintain contact between the rod and the insert's track; translating the bending head within the housing along the y-axis and rotating about its x-axis to maintain position and tangency to the track; and the multi-planar bent rod is removed from the additional insert.

These processes may be repeated multiple times and utilize multiple bending heads. In various embodiments, fewer than all of the recited steps may be followed.

What is claimed is:

1. An apparatus for bending a rod, comprising:
at least one bending die, wherein the at least one bending die can rotate about at least its y-axis;
at least one bending head;
wherein the at least one bending head has a substantial y-axis height compared to the x-axis dimension;
a bending head housing for each of the at least one bending heads;
wherein the bending head housing includes a translational element about its x-axis;
an insert that fits around and is secured to the bending die; and
wherein the substantial y-axis height of the bending head allows for the insert to have a track for the rod that is in multiple planes.

2. The apparatus of claim 1, wherein the insert has a track.

3. The apparatus of claim 2, wherein the track is configured to receive a rod.

4. The apparatus of claim 2, wherein the track is configured for a specific patient.

5. The apparatus of claim 1, wherein the bending head housing comprises a rotational element about its x-axis.

6. The apparatus of claim 1 further comprising a rod configured to be bent by the apparatus, wherein the rod is bent by the bending head being forced against the rod and the rotation of the bending die.

7. The apparatus of claim 1, wherein the bending head has a large y height which allows for travel of the track in the y direction.

8. The apparatus of claim 1, wherein at least one of the x position, y position, and/or rotational position of the bending head is controlled during bending.

9. The apparatus of claim 1, wherein the rotation of the bending die is controlled by one or more actuators.

10. The apparatus of claim 1 further comprising a second bending head and second bending head housing.

11. The apparatus of claim 10, wherein the bending heads are controlled by two or more actuators.

12. A machine for bending a rod, comprising:
at least one die;
wherein the at least one die is configured to rotate about at least its y-axis;
at least one head;
at least one head housing for each of the at least one heads;
wherein the head housing includes a translational element about its x-axis; and
an insert that fits around and is secured to the at least one die.

13. The machine of claim 12, wherein the track is configured to receive a rod.

14. The machine of claim 12, wherein the track is configured for a specific patient.

15. The machine of claim 12, wherein the bending head housing comprises a rotational element about its x-axis.

16. The machine of claim 12 further comprising a rod configured to be bent by the apparatus, wherein the rod is bent by the bending head being forced against the rod and the rotation of the bending die.

17. The machine of claim 12, wherein the bending head has a large y height which allows for travel of the track in the y direction.

18. The machine of claim 12, wherein at least one of the x position, y position, and/or rotational position of the bending head is controlled during bending.

19. The machine of claim 12, wherein the rotation of the bending die is controlled by one or more actuators.

20. The machine of claim 13, wherein rod is not circular in cross-section.
